(19) United States
(12) Patent Application Publication
(10) Pub. No.: US 2022/0287556 A1
(43) Pub. Date: Sep. 15, 2022

(54) THERMAL CONTROL OF IMAGING SYSTEM

(71) Applicant: NEW VIEW SURGICAL INC., Boston, MA (US)

(72) Inventor: Vivek Sikri, Cambridge, MA (US)

(21) Appl. No.: 17/770,584

(22) PCT Filed: Oct. 20, 2020
(86) PCT No.: PCT/US2020/056423

§ 371 Date: Apr. 20, 2022

Related U.S. Application Data
(60) Provisional application No. 62/923,764, filed on Oct. 21, 2019.

(51) Int. Cl. A61B 1/12; A61B 1/06; A61B 17/34; A61B 1/045; A61B 1/00; A61B 1/313

(52) U.S. Cl. CPC A61B 1/128; A61B 1/0655; A61B 17/3421; A61B 1/045; A61B 1/00183; A61B 1/3132

(57) ABSTRACT

The present disclosure includes systems and methods for improving images provided by imaging systems by controlling the temperature of these imaging systems without reducing the image quality or increasing noise in the system. In one aspect, a cannula assembly comprises a processor, and a cannula having a distal end configured for insertion into a patient and housing an imaging device, an image sensor, and a temperature sensor. The processor may be housed within one portion of the tube, or it may be located external to the tube or even external to the patient. The processor is operable to receive temperature information from the temperature sensor, determine, based on the temperature information, whether a temperature of the image sensor is within a predetermined temperature range, and maintain the temperature of the image sensor within the predetermined temperature range by modifying an illumination level of the imaging device.

### CROSS-REFERENCE TO RELATED APPLICATIONS

This application claims the benefit of U.S. Provisional Application Ser. No. 62/923,764, filed Oct. 21, 2019, the entire disclosure of which is incorporated herein by reference for all purposes.

### BACKGROUND

The field of the present disclosure relates generally to systems and methods for improving images provided by imaging systems, and more particularly to cannula assemblies having integrated imaging and illumination devices and being capable of controlling the temperature of these devices.

In minimally invasive surgery, there are often several small incisions made into the body to insert surgical tools, insufflation devices, endoscopes or other viewing devices. Endoscopic surgical procedures performed through a tubular cannula have evolved over the years. Presently, surgeons are performing endoscopic procedures in any hollow viscus of the torso body area after the region has been insufflated. Typically, multiple narrow cannulas are each inserted through individual small entrance wounds (i.e., ports) in the skin in order to accommodate various instruments, as well as different viewing angles. To accomplish their insertion, separate trocars are used in conjunction with the cannulas to puncture the body cavity. A trocar is a guide placed inside the cannula with either a pointed cutting blade, sharpened tip or a blunt tip, depending on whether it is used to puncture the skin or enter through a separately made incision. Once the cannula is inserted, the trocar is removed, leaving the hollow cannula in place for use during the procedure.

Surgeons are now doing procedures in a manner that minimizes the number of incisions to lessen trauma to the patient, reduce the incidence of infection, improve recovery time and decrease cosmetic damage. In certain cases, surgeons would prefer to only have one incision, referred to as Single Port Incision or Single Point Access (SPA). Surgeons are also using natural orifices, such as the mouth, to provide access for procedures using no incision or only incisions internal to the body.

The entry and deployment of imaging and/or lighting components can aid surgical procedures, such as endoscopic procedures. To minimize the number of access ports, cannulas with integrated imaging and lighting components have been developed. Examples of tubular cannula or catheters with deployable imaging and/or lighting components are described in U.S. Pat. No. 5,166,787 to Ilion, U.S. Pat. No. 8,439,830 to McKinley, U.S. Pat. No. 8,052,607, US Patent Application No. 2009/0275799 to Sadat, US Patent Application No. 2009/0259097 to Thompson and US Patent Application No. 2008/0065099 to Cooper, and US Patent Nos. 2003/0032863 and 2007/0238931, the complete disclosures of which are hereby incorporated herein by reference in their entirety for all purposes.

Previous tubular cannula that incorporate integrated imaging and/or lighting components have certain drawbacks. For example, constant illumination of the light sources during a surgical procedure may increase the temperatures of these devices. In some cases, the light sources in these devices may reach temperatures that cause a reduction in the image quality, increase noise in the overall system and/or cause heat damage, thereby decreasing the reliability and operational life of the system.

Accordingly, while the new have proven highly effective and advantageous, still further improvements would be desirable. In general, it would be desirable to provide improved cannula assemblies with integrated imaging and illumination devices that are capable of controlling the temperature of these devices without reducing the image quality for the user.

### SUMMARY

The following presents a simplified summary of the claimed subject matter in order to provide a basic understanding of some aspects of the claimed subject matter. This summary is not an extensive overview of the claimed subject matter. It is intended to neither identify key or critical elements of the claimed subject matter nor delineate the scope of the claimed subject matter. Its sole purpose is to present some concepts of the claimed subject matter in a simplified form as a prelude to the more detailed description that is presented later.

The present disclosure provides systems and methods for improving images provided by imaging systems by controlling the temperature of these imaging systems without reducing the image quality or increasing noise in the system. In one aspect, a cannula assembly comprises a tube having a distal end portion configured for insertion into a patient and housing an imaging device, an image sensor, and a temperature sensor. The assembly further includes a processor coupled to the tube. The processor may be housed within one portion of the tube, or it may be located external to the tube or even external to the patient. The processor is operable to receive temperature information from the temperature sensor, determine, based on the temperature information, whether a temperature of the image sensor is within a predetermined temperature range, and maintain the temperature of the image sensor within the predetermined temperature range by modifying an illumination level of the imaging device.

In one embodiment, the processor is operable to decrease the illumination level in response to the temperature exceeding a high temperature threshold. This ensures that the imaging device does not exceed a maximum temperature during use. In certain embodiments, the processor is operable to increase the exposure of images generated by the image sensor in response to decreasing the illumination level. In other embodiments, the processor is operable to increase a gain of the image sensor in response to decreasing the illumination level. This ensures that the image quality produced by the imaging device remains substantially the same as the illumination level is decreased.

In another embodiment, the processor is operable to increase the illumination level in response to the temperature of the image sensor being less than a low temperature threshold. This ensures that the imaging device does not reach a temperature that is too cool for effective operation. In certain embodiments, the processor is operable to decrease exposure of images generated by the image sensor in response to increasing the illumination level. In other embodiments, the processor is operable to decrease a gain of the image sensor in response to increasing the illumination level.

In yet another embodiment, the processor is operable to both increase the illumination level in response to a temperature of the image sensor being less than the low temperature threshold and to decrease the illumination level when the temperature of the image sensor reaches the high temperature threshold. This ensures that the imaging device remains within a predetermined temperature range during operation.

In certain embodiments, the distal end of the tube further comprises a tip adapted for insertion through a percutaneous penetration and into a body cavity of a patient. In some embodiments, the tip will be configured to create the percutaneous penetration and in other embodiments, the tip will be configured to pass through an opening that has already been formed in the patient. For example, the tip may be formed into a pointed tip, blunt tip or conical tip that is configured to puncture the patient's skin and pass through the incision created by the puncture. In other embodiments, the tube can be fitted with a retractable and/or removable trocar for creation of the incision or percutaneous penetration. The blunt tip may include side sections or fins extending radially outward from the distal surface to facilitate access through an incision and/or to reduce the force necessary to create the incision.

The cannula assembly may further comprise a deployable housing rotatably coupled to the tube between a closed position and one or more open positions. In an exemplary embodiment, the imaging device and the image sensor are housed within the deployable housing. The temperature sensor is preferably located in operational proximity to the imaging device, which may include, for example, a camera with a lens.

In another aspect of the invention, a system comprises a processor and a computer-readable data storage device comprising program instructions. The program instructions, when executed by the processor, control the system to receive temperature information from a temperature sensor of an imaging unit, determine, based on the temperature information, whether a temperature of the imaging unit is within a predetermined temperature range, and maintain the temperature of the imaging unit within the predetermined temperature range by modifying an illumination level of a light source at the imaging device.

In certain embodiments, the system further comprises a surgical device used in a laparoscopic system. The processor may be housed within the surgical device, or it may be operably coupled to the surgical device by connectors, such as wires, or it may be wirelessly coupled to the surgical device. For example, wireless electrical signals can be transferred from the processor to the surgical device with radio waves (e.g., Bluetooth), acoustic energy, infrared or ultrasonic remote control, free-space optical communication, electromagnetic induction and the like. In an exemplary embodiment, the imaging device comprises a cannula assembly including an imaging device and an image sensor. The cannula assembly may comprise a tube with a distal end configured for insertion into a patient and a housing on the tube for housing the imaging device and the image sensor.

In yet another aspect of the invention, a method comprises receiving, by a processor, temperature information from a temperature sensor of an imaging unit, determining, by the processor, based on the temperature information, whether a temperature of the imaging unit is within a predetermined temperature range and maintaining, by the processor, the temperature of the imaging unit within the predetermined temperature range by modifying an illumination level of a light source at the imaging unit. The processor may be housed within the surgical device, or it may be operably coupled to the surgical device by connectors, wires, or wirelessly. In an exemplary embodiment, the imaging device comprises a cannula assembly including an imaging device and an image sensor.

In certain embodiments, the method further comprises receiving images from an image sensor of the imaging unit and normalizing the images based on the modifying of the illumination level of the light source. The method may include decreasing the illumination level in response to the temperature of the imaging unit exceeding a high temperature threshold. The method may also include increasing the illumination level in response to the temperature of the imaging unit being less than a low temperature threshold.

It is to be understood that both the foregoing general description and the following detailed description are exemplary and explanatory only and are not restrictive of the disclosure. Additional features of the disclosure will be set forth in part in the description which follows or may be learned by practice of the disclosure.

### BRIEF DESCRIPTION OF THE DRAWINGS

The accompanying drawings, which are incorporated in and constitute a part of this specification, illustrate several embodiments of the disclosure and together with the description serve to explain the principles of the disclosure.

FIG. 1 depicts a schematic perspective view of an illustrative cannula assembly for use with the systems and methods of the present disclosure;

FIG. 2 depicts a schematic perspective view of the cannula assembly of FIG. 1 in one of its open positions;

FIG. 3 is a side view of a distal portion of the cannula assembly illustrating an integrated camera/lighting assembly according to the present invention;

FIG. 4 is a side view of the cannula assembly of FIG. 3 illustrating a mirror/light path according to the present invention;

FIG. 5 shows a system block diagram illustrating an example of an environment for implementing systems and processes in accordance with aspects of the present disclosure.

FIG. 6 shows a block diagram illustrating an example of a controller for a system in accordance with aspects of the present disclosure.

FIG. 7 shows a flow block diagram illustrating an example of a process performed by a system in accordance with aspects of the present disclosure.

### DETAILED DESCRIPTION

This description and the accompanying drawings illustrate exemplary embodiments and should not be taken as limiting, with the claims defining the scope of the present disclosure, including equivalents. Various mechanical, compositional, structural, and operational changes may be made without departing from the scope of this description and the claims, including equivalents. In some instances, well-known structures and techniques have not been shown or described in detail so as not to obscure the disclosure, Like numbers hi two or more figures represent the same or similar elements. Furthermore, elements and their associated aspects that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment. Moreover, the depictions herein are for illustrative purposes only and do not necessarily reflect the actual shape, size, or dimensions of the system or illustrated components.

The present disclosure relates generally to imaging systems and, more particularly, to controlling heat in imaging systems. Systems and methods in accordance with aspects of the present disclosure operate to control heat in an apparatus integrating imaging and illumination devices together. In some embodiments, a system combines an imaging unit that houses an imaging device, an illuminating device, and a temperature sensor in a compact housing. For example, the imaging unit can be a surgical tool, such as those described herein.

Noise included in images output by the imaging device may increase in proportion to its temperature. In accordance with aspects of the present disclosure, the system can dynamically maintain the temperature of the imaging device within a desired operating range by modifying an illumination level of the illuminating device based on information provided by the temperature sensor. Additionally, the system can dynamically control the gain of the image sensor and/or exposure of an images in response to the illumination level of the illuminating device. By controlling the temperature of the imaging unit, implementations of the disclosed system improve the quality of images output by the image sensor by optimizing the dynamic range of the image sensor and minimizing noise in images generated by the image sensor. Additionally, by dynamically increasing the gain of the image pre-emptively, implementations of the disclosed system enables the reduction of the illumination level and thereby avoid generating high temperatures. Further, by reducing heat, the system can avoid heat damage that may decrease the reliability and operational life of the system.

In a non-limiting example, some implementations can be a laparoscopic system, including a controller and an imaging unit. The imaging unit can be a cannula having a distal end including a lens, a light source, an image sensor, and a temperature sensor. The light source can be a device that emits light in proportion to a drive signal from the controller. For example, the light source can be a light-emitting diode (LED). The image sensor can be a device that detects light reflected from the light source. For example, the image sensor can a camera including a charged coupled device (CCD) and a digital signal processor (DSP). The temperature sensor can be a device that detects a temperature at the distal end of the cannula and output a signal indicative of the temperature. For example, the temperature sensor can be a thermocouple.

The controller can be communicatively connected to the imaging unit and a display device. The controller can include a combination of hardware and software that determines a temperature of the distal end of the cannula based on a temperature signal from the temperature sensor. Using the temperature signal, the controller can automatically control the illumination of the light source to dynamically maintain the temperature of the distal end within a predefined temperature range. More specifically, in response to the temperature exceeding a predetermined high threshold, the controller can reduce the illumination level of the light source until the temperature is equal to or less than the high threshold. And, in response to the temperature falling below a predetermined low threshold, the controller can increase the illumination of the light source until the temperature is equal to or greater than the low threshold. Additionally, in some implementations, the controller can automatically control the gain of the image sensor and/or exposure of the images generated by the sensor based on a brightness of images generated by the image sensor. More specifically, in response to the changes in illumination by the light source, the controller can automatically increase or decrease the gain and/or exposure to maintain the image brightness at a predefined brightness level or a predefined brightness range. Further, the controller can provide a display signal to a display device for viewing by a user.

It is understood that decreasing the illumination and increasing the gain and/or exposure may increase noise of the images output by the disclosed system. However, in accordance with aspects of the present disclosure, maintaining the temperature of the image device within the predefined range reduces dark current of the image sensor, while improving its dynamic range. On the other hand, it is also understood that increasing the illumination and decreasing the gain may increase temperature, which can increase dark current and decrease the usable dynamic range of the image sensor. However, maintaining the temperature of the image device within the predefined range reduces noise in the displayed image. Hence, implementations of systems and methods disclosed herein overcome the heat issues of imaging systems by optimizing noise, dark current, and dynamic range.

While the present disclosure generally describes the controller as separate unit from the image unit, it is understood that some or all of the functionality of the controller can be implemented in the imaging unit and that the entire system can be implemented in a single unit and/or within a signal housing.

Reference will now be made in detail to specific implementations illustrated in the accompanying drawings. In the following detailed description, numerous specific details are set forth to provide a thorough understanding of the disclosure. However, it will be apparent to one of ordinary skill in the art that implementations may be practiced without these specific details. In other instances, well-known methods, procedures, components, circuits, and networks have not been described in detail so as not to unnecessarily obscure aspects of the implementations.

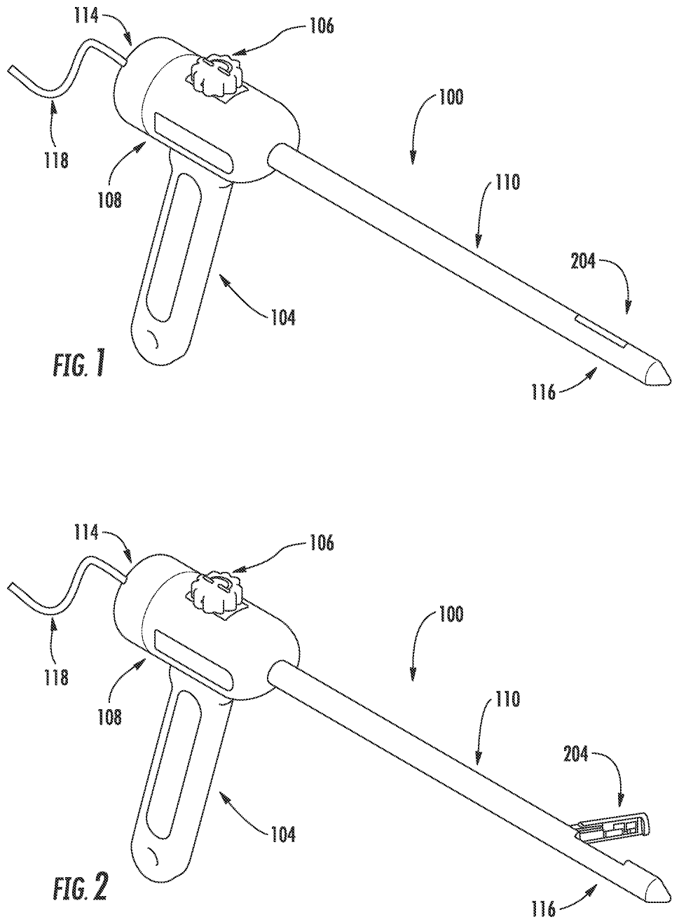

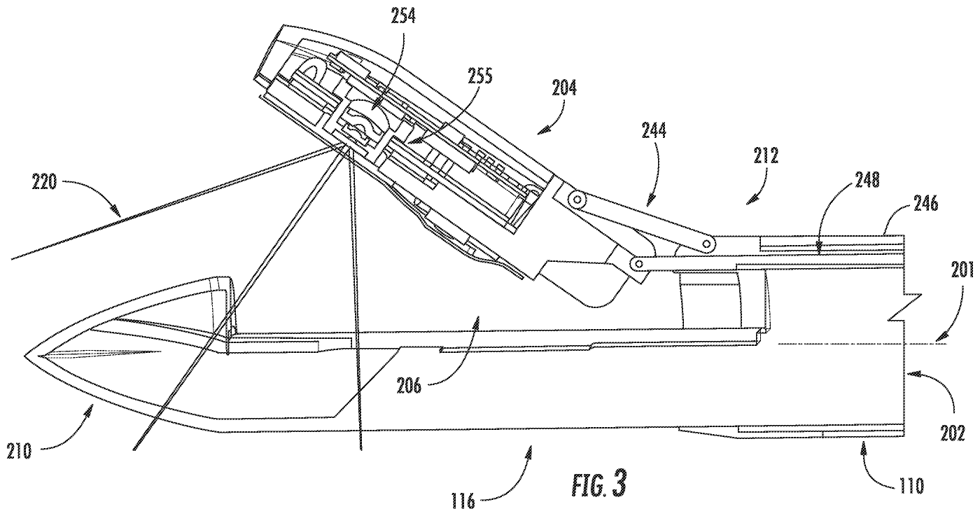

FIGS. 1 and 2 illustrate one embodiment of an illustrative cannula assembly 100 that may be used with the systems and methods of the present disclosure. However, it will be recognized that the present disclosure is not limited to the specific cannula assembly described herein. For example, the systems and methods for controlling temperature may be used with other surgical devices, such as trocars, endoscopes, capsule endoscopes, catheters, indwelling or implantable devices, and the like.

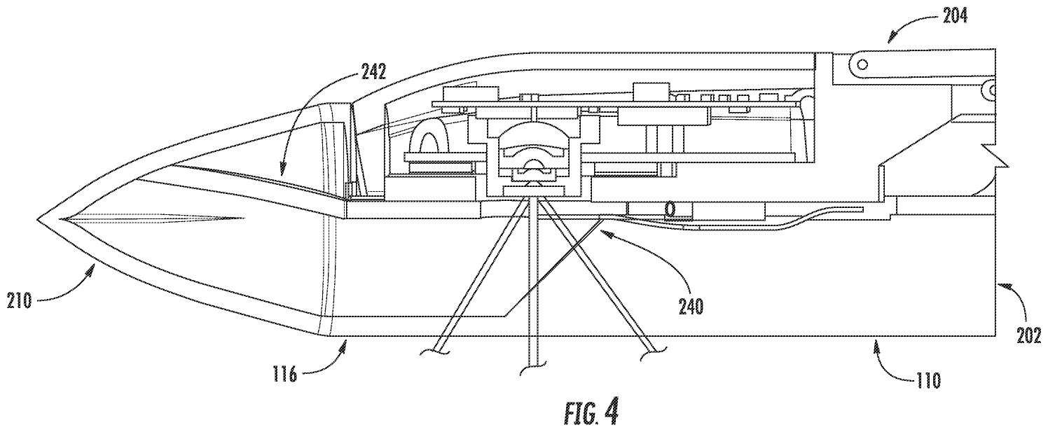

As shown, cannula assembly 100 includes a tube 110 forming an internal lumen 202 (see FIG. 3). A proximal end portion 114 of tube 110 can be adapted for manipulation by the surgeon or clinician, and a distal end portion 116 can be adapted for insertion through a percutaneous penetration and into a body cavity of a patient. In some embodiments, distal end 116 will be configured to create the percutaneous penetration and in other embodiments, distal end 116 will be configured to pass through an opening that has already been formed in the patient. For example, distal end 116 may be formed into a pointed tip, blunt tip or conical tip that is configured to puncture the patient's skin and pass through the incision created by the puncture. In other embodiments, lumen 202 of tube 110 can be fitted with a retractable and/or removable trocar (see, for example, distal end portion 116 in FIG. 3) for creation of the incision or percutaneous penetration. The blunt tip may include side sections or fins extending radially outward from the distal surface to facilitate access through an incision and/or to reduce the force necessary to create the incision. One example of a suitable blunt tip distal end for use with the present invention is an obturator described in U.S. Pat. No. 7,758,603, the complete disclosure of which is hereby incorporated by reference in its entirety for all purposes. Other suitable distal end portions for use in the present invention can be found in U.S. Pat. Nos. 8,940,009 and 6,478,806, the complete disclosures of which are hereby incorporated by reference in their entirety for all purposes.

Cannula assembly 100 further includes a housing 108 having a handle 104 attached near or at proximal end 114 of tube 110 for manipulation by the surgeon or the clinician. Tube 110 may be formed of a variety of cross-sectional shapes, e.g., generally round or cylindrical, ellipsoidal, triangular, square, rectangular, and D-shaped (in which one side is flat). One or more portions of tube 110 may be designed to open once inserted into the body cavity.

In one embodiment depicted in FIG. 3, cannula assembly 100 further includes a movable or deployable housing 204 coupled to tube 110 and designed to open and close relative to the remaining portions of tube 110. Housing 204 may be integral with tube 110 or it may be formed as a separate component that is coupled to tube 110. In either event, housing 204 is disposed on, or coupled to, tube 110 at a position proximal to distal end 116 and distal to proximal end 114. In the preferred embodiment, housing 204 resides far enough along tube 110 in the distal direction such that it is positioned within the body cavity of the patient during use. At the same time, housing 204 is positioned far enough proximal to distal end 116 such that it does not interfere with the insertion of distal end 116 of tube 110 as distal end 116 is passing the percutaneous penetration or incision in the patient. In addition, housing 204 is positioned proximally from distal end 116 to protect the electronic components therein (discussed below) as distal end 116 creates an incision and/or passes through an existing incision in the patient.

All or parts of cannula assembly 100 are capable of being positioned into the closed position for insertion and extraction either directly into the body cavity or through another insufflating cannula. In certain embodiments, tube 110 comprises an internal lumen 202 that can be fitted with a retractable and/or removable trocar. In one embodiment, the trocar is made of solid, non-transparent material. In another embodiment, all or parts of the trocar are made of optically transparent or transmissive material such that the trocar does not obstruct the view through the camera (discussed below).

Cannula assembly 100 further comprises an actuator mechanism that includes a proximal control 106 for moving housing 204 between the closed position (FIG. 1) and the open position (FIG. 2). Alternatively, proximal control 106 can incrementally move housing 204 between any number of positions between the open and closed positions. Proximal control 106 may be situated on handle 108 as shown in FIGS. 1 and 2, or it may be part of a robotic control system that is remotely controlled by an operator.

Housing 204 houses an electronic component, which is at least partially disposed within tube 110 when in the closed position. In certain embodiments, lumen 202 is substantially free from obstruction by the electronic components of housing 204 when in the closed position. This allows various instruments, e.g., surgical tools or other electronic components, to be passed through lumen 202 and used during the operation or surgical procedure. In other embodiments, the electronic components of housing 204 may partially obstruct lumen 202 in the closed position, but will not obstruct lumen 202 in the open position or at least some of the positions between the fully open and closed positions.

As shown in FIG. 3, the electronic components include one or more image transmission components 254, in combination with one or more illumination components 255. In one embodiment, image transmission component 254 may be a charge-coupled device (CCD) camera, a complementary metal oxide semi-conductor (CMOS) imaging device, and/or an imaging fiber optic cable and their ancillary optics and electronic drivers for power, communication and other functions. Optically, one or more of the image transmission components 254 may also image across the spectrum, including those portions invisible to the human eye, such as infrared and ultra-violet. In one embodiment, two image transmission components may be configured to capture stereoscopic images (in still and/or in motion). In one embodiment, one or more of the image transmission components 254 may be configured with any of a combination of fixed optics, adaptive optics, and/or active optics. Adaptive and active optics can be capable of focusing and/or zooming onto the image or target area.

In one embodiment, the one or more image transmission components 254 are capable of capturing both motion and still images, and transmitting them to the surgeon or operator through wired or wireless communication device 118 housed within or connected to the housing 108, handle 104, lumen 202 and/or the tubular element 110 wall. Such communication devices 118 may include electrical signals, such as analog and/or digital, or a fiber communication system.

The illumination component 305 may be one or more light or illumination sources and their ancillary electronic drivers. In one embodiment, the illumination sources are Light Emitting Diodes (LED), organic LED (OLED), illumination fiber optic, filament lamps, electroluminescent and/or laser sources. In certain embodiments, the illumination component 255 is tailored to work closely in both optical and spectrum characteristics with the image transmission component 254, with the illumination area, level and homogeneity being optimized. In one example, this may mean the illumination level is controlled by the surgeon or clinician; whereas, in another embodiment, Automated Gain Control (AGC) is correlated with the illumination level of the illumination component 255. A more complete description of suitable illumination and image transmission components can be found in U.S. Pat. No. 8,439,830 to McKinley, the complete disclosure of which is hereby incorporated by reference in its entirety for all purposes.

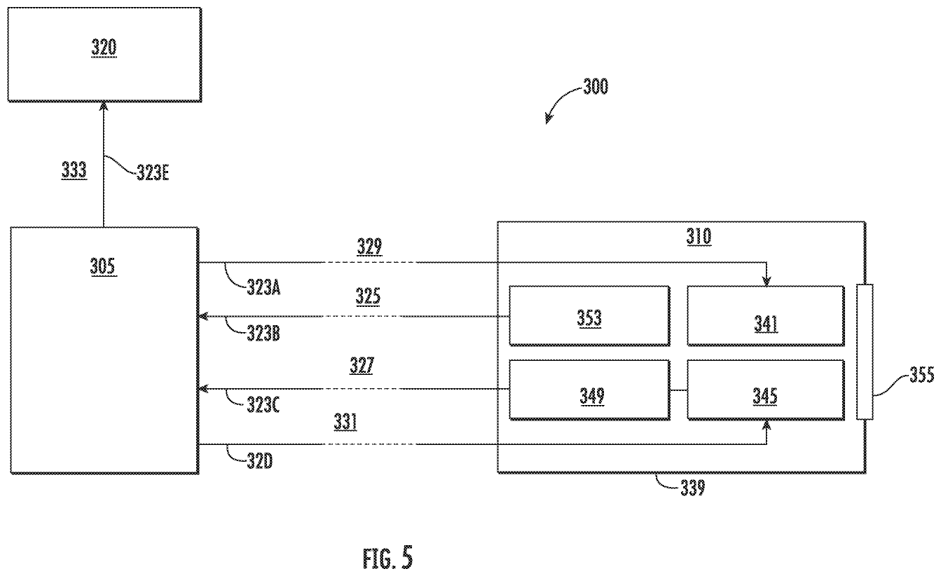

Cannula assembly 100 further includes a temperature sensor, an image sensor and an image processor (not shown) as further described in reference to FIGS. 5-7. These elements are coupled to image transmission component 254 and illumination component 255 and may be disposed in deployable housing 204, or in another location of shaft 110. A controller (not shown) is also coupled to the temperature sensor, image sensor and the image processor, as described in further detail below. The controller may be disposed within housing 204, in another location of shaft 110, within handle 104 or it may be disposed externally to cannula assembly 100 and coupled thereto with wired connectors or wirelessly through devices known by those skilled in the art.

Referring now to FIGS. 3 and 4, a preferred embodiment of cannula assembly 100 will now be described (note that these figures only show the distal portion of cannula assembly 100). In this embodiment, distal end portion 116 is a separate component, such as a trocar, obturator or the like, that is removably coupled to tube 110 such that the distal end portion 116 may be translated in the longitudinal direction relative to tube 110 through lumen 202. For example, prior to insertion, housing 204 is opened and distal end portion 210 is translated through inner lumen 202 until it passes distally of the end of tube 110. Housing 204 is then closed and the obturator 116 can be used to create an incision, enlarge an incision or pass through an incision or other opening in the patient (as shown in FIG. 4). After tube 110 and housing 204 have been inserted through the incision and into the patient, housing 204 may be opened and distal end 116 retracted through internal lumen 202 of tube 110. Removal of distal end or obturator 116 provides an open internal lumen 202 to allow for the passage of instruments, tissue or the like through tube 110 during the surgical procedure.

In this embodiment, distal end 116 of tube 110 has a substantially conical outer surface that extends to a relatively sharpened distal tip 210. However, it will be understood that distal end 116 may comprise a variety of different shapes and sizes, such as a substantially cylindrical or rectangular surface or a blunt end. Housing 204 is coupled to a distal end of tube 110 and sized to fit between distal tip 210 and tube 110 when housing 204 is in the closed position and distal tip 210 has been translated distally of tube 110, as show in FIG. 3. Of course, it will be recognized that distal end portion or obturator 116 may be integral with tube 110. In this embodiment, movable housing 204 is preferably sized to fit within a compartment 206 of tube 110 proximal to distal end 116 when in the closed position.

Housing 204 is preferably spaced away from distal tip 210 a sufficient distance to protect the electronic components therein as distal tip 210 is deployed to create and/or pass through an incision in the patient, or as tube 110 is maneuvered within a body cavity of the patient. In an exemplary embodiment, the proximal end of housing 204 is spaced at least about 5 mm to about 50 mm from the end of distal tip 210, preferably about 10 mm to about 40 mm, and more preferably about 20-30 mm.

Housing 204 is pivotally coupled to tube 110 via a hinge 212 that allows housing 204 to be pivoted away from tube 110 through a variety of different orientations between the closed and open positions. As shown, this provides the surgeon or operator with the ability to effectively “triangulate” one or more fields of views of the image transmission component and the illumination component. Adjusting the angle of the opening of deployable housing 204 relative to the longitudinal axis 201 of tube 110 causes the direction of view 220 to be adjusted without movement of the cannula. This allows the view to be changed slightly, without reverting to the need to move the cannula. In use, tube 110 may be rotated around axis 201 so that the image transmission and illumination components cover more fields of use. Alternatively, deployable housing 204 may be pivoted about more than one axis such that the direction of view can be lateral relative to axis 201 or even proximal along axis 201, as described below in reference to FIG. 11.

All or a portion of distal tip 210 of obturator 116 may be formed from an optically transparent material to allow the surgeon to see a forward view beyond distal end 116 (i.e., along axis 201 of tubular element 110). Tube 110 further includes an opening between housing 204 and inner lumen 202 of tube 110 to allow light from the image transmission and illumination sources to pass through. Cannula assembly 100 preferably includes one or more reflective surfaces 240, such as mirrors or the like, positioned at an angle relative to axis 201 such that the light emitted from image transmission components 304 and/or illumination components 305 reflects off surface(s) 240 and passes distally through distal tip 210. The reflective surface 240 may be coupled to a rod or other suitable connection (not shown) that passes through lumen 202 to proximal end 114, allowing surface 240 to be retracted from tube 110 once deployable housing 204 is opened, if necessary. Alternatively, reflective surface 240 may be part of the obturator 116, which is removed during operation.

Cannula assembly 100 further includes a substantially opaque surface or wall 242 extending to an internal surface of distal tip 210. Opaque wall 242 blocks light from the illumination elements 255 from passing directly into lumen 202 or distal end 116 (other than through opening 238) such that the light does not interfere with the image transmission components 304. This provides a much clearer view of the surgical field when the device is in the closed position and the surgeon is viewing forward along axis 201.

Referring again to FIG. 3, hinge 212 comprises a link 244 coupled to an upper surface of housing 204 and pivotally coupled to an outer surface 246 of tube 110 via pins or other suitable hinges. A push rod 248 is disposed within tube 110 and coupled to housing 204 and a proximal control knob 106 on handle 104 (see FIG. 1). Operation of knob 106 causes push rod 248 to translate distally or proximally. Distal translation of rod 248 forces housing 204 forward such that it pivots about link 244 into one or more open positions. In this configuration, imaging component 254 and illumination component 255 face the area of interest. The angle of opening of housing 204 may be adjusted by the amount of rod 248 fed into tube 110 by rotation of knob 106. This arrangement allows for the image and illumination components 254, 255 to occupy a portion of lumen 202 in the closed position, and to leave lumen 202 substantially open and available for instrument insertion, operation and/or removal when open. In addition, this arrangement protects the image and illumination components 254, 255 when closed.

The cannula assembly of the present invention is not limited to the specific device shown in FIGS. 1-4. A more complete description of various embodiments of the cannula assembly can be found in commonly-assigned, co-pending U.S. application Ser. No. 16/508,738, filed Jul. 11, 2019, the complete disclosure of which is incorporated herein by reference in its entirety for all purposes.

FIG. 5 shows a block diagram illustrating an example of an environment 300 for implementing systems and methods in accordance with aspects of the present disclosure. The environment 300 can include a controller 305, an imaging unit 310, and a display 320. The controller 305 can be a computing device connected to the imaging unit 310 and the display 320 through one or more wired or wireless communication channels 323A, 323B, 323C, 323D, and 323E, which may use various serial, parallel, video transmission protocols suitable for their respective signal types. For example, the communication channel 323A can be an Inter-Integrated Circuit (IIC) bus providing the light control signal 329. The controller 305 can include hardware, software, or a combination thereof for performing operations in accordance with the present disclosure. The operations can include receiving a temperature signal 325 and an image signal 327 from the imaging unit 310. The operations can also include processing the temperature signal 325 to determine whether it is within a predetermined operating range. The operations can also include dynamically modifying the light control signal 329 based on the processing of the temperature signal 325 to maintain the temperature of the imaging unit 310 within the predetermined temperature range. The operations can further include processing the image signal 327 based on the current illumination of the imaging unit 310 to dynamically modify the exposure and/or gain of images in the display signal 333 to optimize the amount of noise in the images.

The imaging unit 310 can include a one or more devices that generate light for illuminating an area. In some implementations, the imaging unit 310 is a surgical tool, as previously described herein. For example, the imaging unit 310 can be a distal end of a laparoscopic tool used to illuminate a body cavity and record images inside the body cavity. As illustrated in FIG. 5, the imaging unit 310 can comprise a housing 339 enclosing a light source 341, an image sensor 345, image processor 349, and a temperature sensor 353, and a lens 355. The light source 341 can be dimmable light-emitting device, such as a LED, a halogen bulb, an incandescent bulb, or other suitable light emitter. The dimming of the light source 341 can be controlled by the light control signal 329, which may control the dimming via, for example, a variable voltage, a variable current, pulse-width modulation or IIC messages.

The image sensor 345 can be a device configured to detect light reflected from the light source 341 and outputs video information, including image data, to the image processor 349. The image sensor 345 can be a CCD or other suitable imaging device. The image processor 349 can be a device configured to receive the video information from the image sensor 345, condition the image data, and output the image signal 327 including the conditioned image data. In accordance with aspects of the present disclosure, conditioning the image data can include normalizing the exposure of images included in the image data. In some implementations, the image processor 349 can also control the gain of the image sensor 345 based on the received image data to normalize the images in response to modification of the illumination by the light source 341.

The temperature sensor 353 can be a device configured to output the temperature signal 325 corresponding to the temperature inside the housing 339 of the imaging unit 310. The temperature sensor 353 can be, for example, a thermocouple or other suitable temperature-sensing device. In some implementations, the temperature sensor 353 can be a separate device. In other implementations, the temperature sensor 353 can be combined with another device, such as the light source 341, the image sensor 345, and the image processor 349. For example, the temperature sensor 353 can be part of the image sensor 345. The temperature sensor 353 can output the temperature signal 325 as, for example, a variable voltage signal, a variable current signal, pulse-width modulated signal, or an IIC message, in response to changes in heat of the imaging unit 310 due to modification of the illumination by the light source 331.

The lens 355 can provide a transparent portal permitting light from the light source 341 to exit the housing 339 and illuminate an area, such as internal body cavity. The lens 355 can also permit reflections of such light to reenter the housing for recording by the image sensor 345 to generate images of the area.

While FIG. 5 illustrates the temperature signal 325, the image signal 327, light control signal 329, and the image control signal 331 as being communicated using communication channels 323A, 323B, 323C, 323D, respectively, it is understood that one or more of the temperature signal 325, the image signal 327, light control signal 329, and the image control signal 331 can be combined into one communication channel. In some implementations, some or all of the temperature signal 325, the image signal 327, light control signal 329, and the image control signal 331 can be combined onto a single communication channel 323A. For example, one or more of the communication channels 323A, 323B, 323C, 323D can be combined into a bus line.

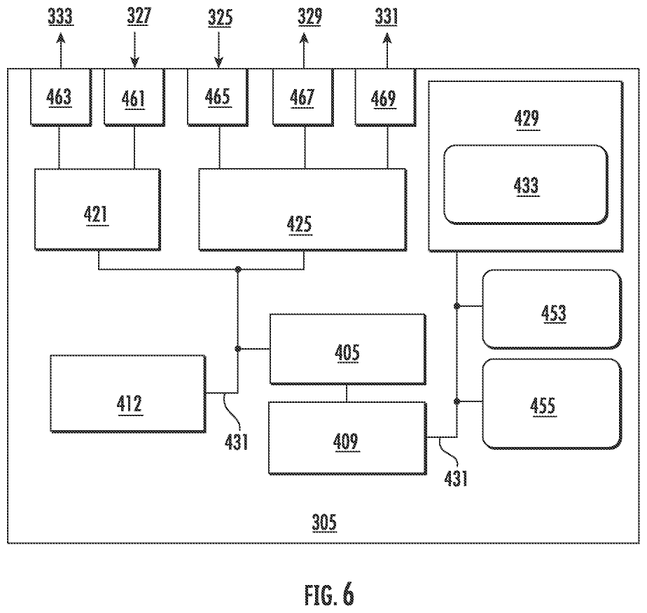

FIG. 6 shows a functional block diagram illustrating a controller 305 in accordance with aspects of the present disclosure. The controller 305 can be the same or similar to that previously describe herein. The controller 305 can include a processor 405, a memory device 409, a network interface 413, an image processor 421, an I/O processor 425, a storage device 429, and a data bus 431. Also, the controller 305 can include image input connection 461, image output connection 463 that receive and transmit video signals from the image processor 421. Further, the controller can include input/output connections 465, 467, and 469 that receive/transmit data signals from I/O processor 425.

In implementations, the processor 405 can include one or more microprocessors, microchips, or application-specific integrated circuits. The memory device 409 can include one or more types of random-access memory (RAM), read-only memory (ROM) and cache memory employed during execution of program instructions. Additionally, the controller 305 can include one or more data buses 431 by which it communicates with the memory device 409, the network interface 413, the image processor 421, the I/O processor 425, and the storage device 429.

The I/O processor 425 can be connected to the processor 405 and may include any device that enables an individual to interact with the processor 405 (e.g., a user interface) and/or any device that enables the processor 405 to communicate with one or more other computing devices using any type of communications link. The I/O processor 425 can generate and receive, for example, digital and analog inputs/outputs according to various data transmission protocols.

The storage device 429 can comprise a computer-readable, non-volatile hardware storage device that stores information and program instructions. For example, the storage device 429 can be one or more, flash drives and/or hard disk drives. In accordance with aspects of the present disclosure, the storage device 429 can store temperature control information 433. The temperature control information 433 can include, for example, a high temperature threshold, a low temperature threshold, and temperature-illumination level maps.

The processor 405 executes program instructions (e.g., an operating system and/or application programs), which can be stored in the memory device 409 and/or the storage device 429. The processor 405 can also execute program instructions of a temperature control module 453 and an image processing module 455. The temperature control module 453 can be configured to determine whether a temperature indicated by a temperature signal 325 is within a predetermined range. Further, based on such determination, the temperature control module 453 can be configured to modify a light control signal 329 to modify the illumination level of a light source (e.g., light source 341) to maintain the temperature of the light source within a desired operating range. The image processing module 455 can be configured to analyze images received in an image signals 327 from an imaging device (e.g., image sensor 345), determine a brightness of the images, and modify the exposure of the images to normalize their brightness.

It is noted that the controller 305 is only representative of various possible equivalent-computing devices that can perform the processes and functions described herein. To this extent, in embodiments, the functionality provided by the controller 305 can be any combination of general and/or specific purpose hardware and/or program instructions. In each embodiment, the program instructions and hardware can be created using standard programming and engineering techniques.

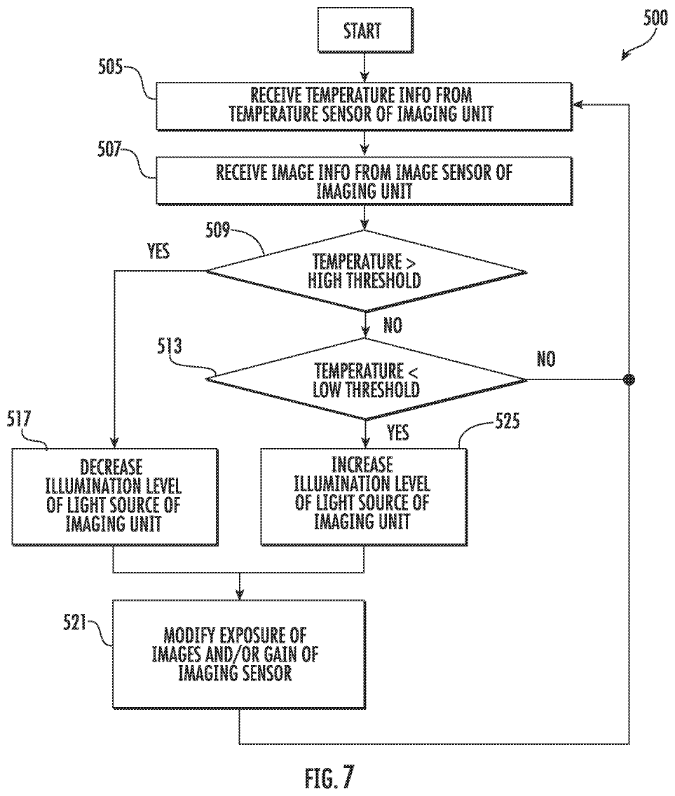

The flow diagram in FIG. 7 illustrates an example of the functionality and operation of possible implementations of systems, methods, and computer program products according to various implementations consistent with the present disclosure. Each block in the flow diagram of FIG. 7 can represent a module, segment, or portion of program instructions, which includes one or more computer executable instructions for implementing the illustrated functions and operations. In some alternative implementations, the functions and/or operations illustrated in a particular block of the flow diagram can occur out of the order shown in FIG. 7. For example, two blocks shown in succession can be executed substantially concurrently, or the blocks can sometimes be executed in the reverse order, depending upon the functionality involved. It will also be noted that each block of the flow diagram and combinations of blocks in the block can be implemented by special purpose hardware-based systems that perform the specified functions or acts, or combinations of special purpose hardware and computer instructions.

FIG. 7 shows a flow block diagram illustrating an example of a process 500 for a system that manages temperature of an imaging unit (e.g., imaging unit 310) to improve image quality by optimizing the dynamic range of an image sensor and image noise, while also improving the reliability of the imaging unit. At block 505, the system (e.g., controller 305 executing temperature control module 453) receives temperature information (e.g., temperature signal 325) from an imaging unit (e.g., imaging unit 310). In some implementations, the system receives the temperature information from a temperature sensor (e.g., temperature sensor 353) located proximal to an image sensor (e.g., image sensor 345) housed (e.g., in housing 339) within the imaging unit. At block 507, the system receives images from the image sensor of the imaging unit. For example, for implementations in which the system is a laparoscopic system, the imaging unit may record images while inserted into a body cavity of a patient that is illuminated by a light source housed with the image sensor in the imaging unit.

At blocks 509 and 513, the system determines whether the temperature information is within a predetermined temperature range. Based on the temperature, the system maintains the temperature of the imaging unit within the predetermined temperature range by dynamically modifying an illumination level of a light source at the imaging unit. For example, the imaging sensor may have an acceptable dynamic range when operating at temperatures between 40 degrees Fahrenheit and 140 degrees Fahrenheit. However, during a laparoscopic procedure, the temperature of the distal end of the imaging unit when inside a body cavity may range in temperature between 95 degrees Fahrenheit and 200 degrees Fahrenheit. Accordingly, implementations consistent with the present disclosure operate to maintain the temperature of the image sensor within the acceptable operating range. That is, at block 509, the system can determine whether the current temperature is greater than a predetermined high threshold value (e.g., stored in temperature control information 433).

In some implementations, the high threshold value may be 140 degrees Fahrenheit. At that temperature, the dynamic range of the image sensor may be substantially limited by the heat. As a result, the images displayed by the system may provide unacceptable detail to a user. If the system determines that the temperature is not greater than the high threshold (e.g., block 509 is “No”), then at block 513, the system can determine whether the current temperature is less than a predetermined low temperature threshold value (e.g., stored in temperature control information 433). In some implementations, the low threshold value may be 40 degrees. At block 513, if the system determines the temperature is not less than the low threshold, then the process 500 iteratively returns to block 505 and repeats blocks 505 to 513 while the current temperature receive in the temperature signal with below the high threshold an above the low threshold.

On the other hand, at block 509, if the system determines that the current temperature is greater than the high threshold, then at block 517, the system decreases an illumination level of the imaging unit. For example, the system can modify a light control signal (e.g., light control signal 329) to lower the illumination level of a light source (e.g., light source 341). In some implementations, the system can modify a drive signal for the light source by reducing its voltage, current, or pulse-width. Further, in some implementations, the system can do so by incrementally reducing the drive signal. And, in other implementations, the system can do so by progressively reducing the drive signal based on a predefined mapping between temperature and drive signal levels (e.g., using a map stored in temperature control information 433). Additionally, at block 521, the system (e.g., executing imaging processing module 455) can increase the exposure and/or gain of images output by the image unit in response to the decreased illumination at block 517. The process 500 can then iteratively return to block 505.

Referring back to block 513, if the system determines that the current temperature is below than the low threshold (e.g., block 513 is “Yes”), then at block 525, the system can increase an illumination level of the imaging unit. For example, the system can modify a light control signal to raise the illumination level of a light source. For example, the system can modify a drive signal for the light source by increasing its voltage, current, or pulse-width. In some implementations, the system can incrementally increase the drive signal. In other implementations, the system can progressively increase the drive signal based on a predefined mapping, as described above. Additionally, at block 521, the system (e.g., executing imaging processing module 455) can automatically modify the gain of the image sensor and the exposure of an images output by the image sensor in response to the modifications of the illumination level of the illuminating device at blocks 517 and 525. The process 500 can then iteratively return to block 505.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.” In addition, where features or aspects of the disclosure are described in terms of Markush groups, those skilled in the art will recognize that the disclosure is also thereby described in terms of any individual member or subgroup of members of the Markush group.

Hereby, all issued patents, published patent applications, and non-patent publications that are mentioned in this specification are herein incorporated by reference in their entirety for all purposes, to the same extent as if each individual issued patent, published patent application, or non-patent publication were specifically and individually indicated to be incorporated by reference.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of presently disclosed embodiments. Thus the scope of the embodiments should be determined by the appended claims and their legal equivalents, rather than by the examples given.

Persons skilled in the art will understand that the devices and methods specifically described herein and illustrated in the accompanying drawings are non-limiting exemplary embodiments. The features illustrated or described in connection with one exemplary embodiment may be combined with the features of other embodiments. Various alternatives and modifications can be devised by those skilled in the art without departing from the disclosure. Accordingly, the present disclosure is intended to embrace all such alternatives, modifications and variances. As well, one skilled in the art will appreciate further features and advantages of the present disclosure based on the above-described embodiments. Accordingly, the present disclosure is not to be limited by what has been particularly shown and described, except as indicated by the appended claims.

1. A cannula assembly comprising:
a tube having a distal end portion configured for insertion into a patient and housing an imaging device, an image sensor, and a temperature sensor; and
a processor coupled to the tube and operable to:
receive temperature information from the temperature sensor;
determine, based on the temperature information, whether a temperature of the image sensor is within a predetermined temperature range; and
maintain the temperature of the image sensor within the predetermined temperature range by modifying an illumination level of the imaging device.

2. The cannula assembly of claim 1, wherein the distal end further comprises a tip operable to create an incision.

3. The cannula assembly of claim 1, wherein the predetermined temperature range comprises a high temperature threshold, and the processor is operable to decrease the illumination level in response to the temperature exceeding the high temperature threshold.

4. The cannula assembly of claim 3, wherein the processor is operable to increase exposure of images generated by the image sensor in response to decreasing the illumination level.

5. The cannula assembly of claim 3, wherein the processor is operable to increase a gain of the image sensor in response to decreasing the illumination level.

6. The cannula assembly of claim 1, wherein the predetermined temperature range comprises a low temperature threshold, and the processor is operable to increase the illumination level in response to the temperature of the image sensor being less than the low temperature threshold.

7. The cannula assembly of claim 6, wherein the processor is operable to decrease exposure of images generated by the image sensor in response to increasing the illumination level.

8. The cannula assembly of claim 6, wherein the processor is operable to decrease a gain of the image sensor in response to increasing the illumination level.

9. The cannula assembly of claim 1, wherein the imaging device comprises includes a camera with a lens.

10. The cannula assembly of claim 1, further comprising a deployable housing rotatably coupled to the tube between a closed position and one or more open positions, wherein the imaging device and the image sensor are housed within the deployable housing, wherein the imaging device is configured to provide a longitudinal view when the deployable housing is in the closed position and a transverse view relative to a longitudinal axis of the tube when the housing is in the one or more open positions.

11. (canceled)

12. (canceled)

13. (canceled)

14. A system comprising:
a processor; and
a computer-readable data storage device comprising program instructions that, when executed by the processor, control the system to:
receive temperature information from a temperature sensor of an imaging unit;
determine, based on the temperature information, whether a temperature of the imaging unit is within a predetermined temperature range; and
maintain the temperature of the imaging unit within the predetermined temperature range by modifying an illumination level of a light source at the imaging device.

15. The system of claim 14, wherein the system is operable to:
receive images from an image sensor of the imaging unit; and
normalize the images based on the modifying of the illumination level of the light source.

16. The system of claim 14, wherein:
the system comprises a surgical device used in a laparoscopic system; and
the imaging device comprises a distal end of the laparoscopic system.

17. The system of claim 14, wherein the imaging device comprises a cannula assembly including an imaging device and an image sensor, wherein the cannula assembly comprises a tube with a distal end configured for insertion into a patient and a housing on the tube for housing the imaging device and the image sensor.

18. (canceled)

19. The system of claim 17, where the housing is rotatably coupled to the tube between a closed position and one or more open positions.

20. (canceled)

21. (canceled)

22. The system of claim 14, wherein the predetermined temperature range comprises a high temperature threshold, and the processor is operable to decrease the illumination level in response to the temperature of the imaging unit exceeding the high temperature threshold.

23. The system of claim 14, wherein the system is operable to increase exposure of images generated by an image sensor in response to decreasing the illumination level.

24. The system of claim 14, wherein the system is operable to increase a gain of an image sensor in response to decreasing the illumination level.

25. The system of claim 14, wherein the predetermined temperature range comprises a low temperature threshold, and the processor is operable to increase the illumination level in response to the temperature of the imaging device being less than the low temperature threshold.

26. The system of claim 25, wherein the system is operable to decrease exposure of images generated by an image sensor in response to increasing the illumination level.

27. The system of claim 25, wherein the system is operable to decrease a gain of an image sensor in response to increasing the illumination level.

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)
